(19) United States
(12) Patent Application Publication
(10) Pub. No.: US 2026/0069883 A1
(43) Pub. Date: Mar. 12, 2026

(54) SYSTEMS AND METHODS FOR ORAL HYGIENE LIGHT THERAPY

(71) Applicant: BioLight, Inc., Hamilton, MT (US)

(72) Inventor: Michael BELKOWSKI, Hamilton, MT (US)

(73) Assignee: Biolight, Inc, Hamilton, MT (US)

(21) Appl. No.: 19/325,677

(22) Filed: Sep. 11, 2025

Related U.S. Application Data
(60) Provisional application No. 63/693,459, filed on Sep. 11, 2024.

(51) Int. Cl. A61N 5/06

(52) U.S. Cl. CPC A61N 5/0603; A61N 5/0624; A61N 2005/0606; A61N 2005/0644; A61N 2005/0651; A61N 2005/0663

(57) ABSTRACT

An oral device including a base, a shaft including a head with one or more triple light emitting diodes (LEDs), wherein the shaft is magnetically coupled to the base to receive magnetically transferred power, and wherein when the shaft is in magnetic contact with the base to receive the magnetically transferred power, the one or more triple light emitting diodes (LEDs) are configured to emit light.

### CROSS REFERENCE TO RELATED APPLICATIONS

This application claims priority to U.S. Provisional Application 63/693,459, filed Sep. 11, 2024, the entire disclosure of which is hereby incorporated by reference in its entirety.

### TECHNICAL FIELD

Various embodiments of the present disclosure relate generally to a light therapy oral hygiene device and, more particularly, to devices configured to provide triple light emitting diode (LED) based red, blue, and/or near-infrared light therapy to the oral cavity of the user using magnetically transferred power.

### BACKGROUND

Traditional oral hygiene devices such as toothbrushes have been used to clean teeth, gums, and the tongue. Some toothbrushes provide teeth whitening. However, there exists a need for providing oral health benefits to users that is not met by the existing toothbrushes.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for using a light therapy oral hygiene device.

Aspects disclosed herein include an oral device comprising: a base; and a shaft including a head with one or more triple light emitting diodes (LEDs), wherein the shaft is magnetically coupled to the base to receive magnetically transferred power; wherein when the shaft is in magnetic contact with the base to receive magnetically transferred power, the one or more triple light emitting diodes (LEDs) are configured to emit light.

Aspects also disclosed herein include a system for providing light therapy to an oral cavity, the system comprising: a base; a shaft magnetically coupled to the base to receive magnetically transferred power; one or more sensors attached to the base or shaft; one or more triple light emitting diodes (LEDs) disposed on a head of the shaft, wherein each triple LED is configured to output red light, near red light, and blue light; at least one memory storing instructions; and at least one processor executing the instructions to perform a process, the processor configured to: receive sensed data sensed by the one or more sensors; receive a oral device configuration based on the sensed data, the oral device configuration comprising one or more of wavelengths of light, intensities of light, rates, durations, or frequencies for configuring oral device; and configure the oral device based on the oral configuration.

It is to be understood that both the foregoing general description and the following detailed description are exemplary and explanatory only and are not restrictive of the disclosed embodiments, as claimed

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts a front view of an oral hygiene device with triple light emitting diodes, according to one or more embodiments.

FIG. 1B depicts a side view of an oral hygiene device with triple light emitting diodes, according to one or more embodiments.

FIG. 1C depicts an exploded view of an oral hygiene device with triple light emitting diodes, according to one or more embodiments.

FIG. 1D depicts a cross-sectional view of an oral hygiene device with triple light emitting diodes, according to one or more embodiments.

FIG. 2A depicts a top view of a charging dock for an oral hygiene device with triple light emitting diodes, according to one or more embodiments.

FIG. 2B depicts a front view of a charging dock for an oral hygiene device with triple light emitting diodes, according to one or more embodiments.

FIG. 2C depicts a bottom view of a charging dock for an oral hygiene device with triple light emitting diodes, according to one or more embodiments.

FIG. 3A depicts a front view of an oral hygiene device with triple light emitting diodes, according to an alternative embodiment.

FIG. 3B depicts a side view of an oral hygiene device with triple light emitting diodes, according to an alternative embodiment.

FIG. 3C depicts a back view of an oral hygiene device with triple light emitting diodes, according to an alternative embodiment.

FIG. 4A depicts a front view of an oral hygiene device with triple light emitting diodes, according to an alternative embodiment.

FIG. 4B depicts a front view of an oral hygiene device with triple light emitting diodes, according to an alternative embodiment.

FIG. 5A depicts a diagram of light penetration from a red light or blue light emitted from a toothbrush with triple light emitting diodes, according to one or more embodiments.

FIG. 5B depicts a diagram of light penetration from a near infrared light emitted from a toothbrush with triple light emitting diodes, according to one or more embodiments.

FIG. 6A depicts a flow chart for targeted light therapy, according to one or more embodiments.

FIG. 6B depicts a system environment for targeted light therapy, according to one or more embodiments.

FIG. 7 depicts a data flow for training a machine learning model, according to one or more embodiments.

FIG. 8 depicts an example system that may execute techniques presented herein.

### DETAILED DESCRIPTION OF EMBODIMENTS

The terminology used herein may be interpreted in its broadest reasonable manner, even though it is being used in conjunction with a detailed description of certain specific examples of the present disclosure. Indeed, certain terms may even be emphasized herein; however, any terminology intended to be interpreted in any restricted manner will be overtly and specifically defined as such in this Detailed Description section. Both the general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

As applied herein, an oral cavity may generally refer to a mouth and may include the areas including, approximate to, or in contact with the lips, the lining inside the cheeks and lips, the front two thirds of the tongue, the upper and lower gums, the floor of the mouth under the tongue, the roof of the mouth, and the area around the wisdom teeth.

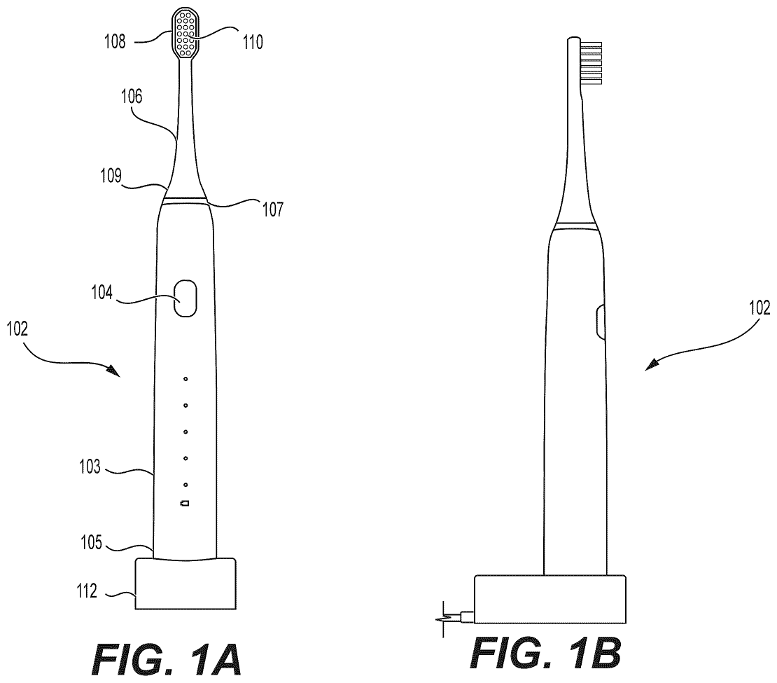

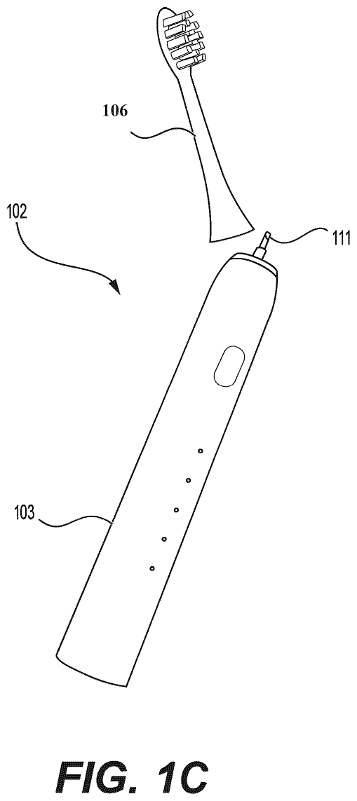

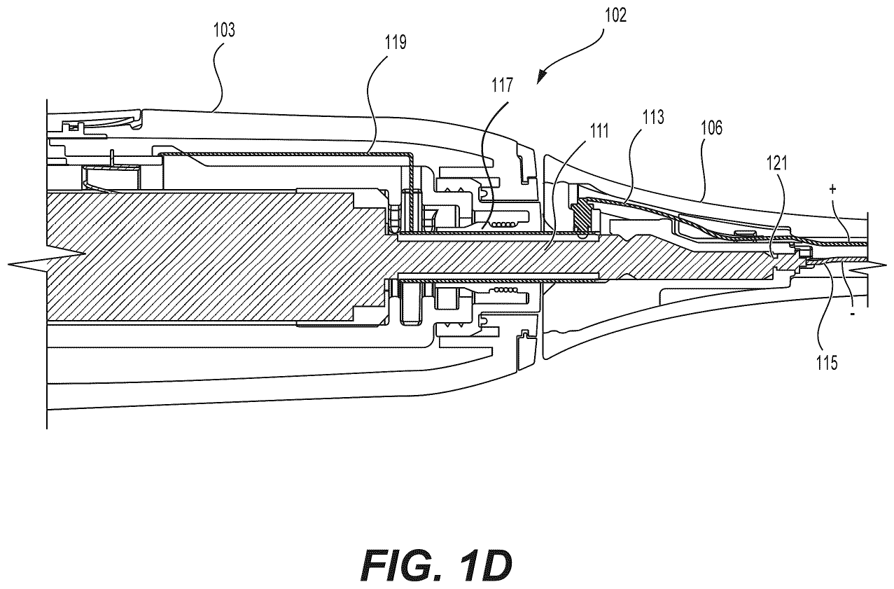

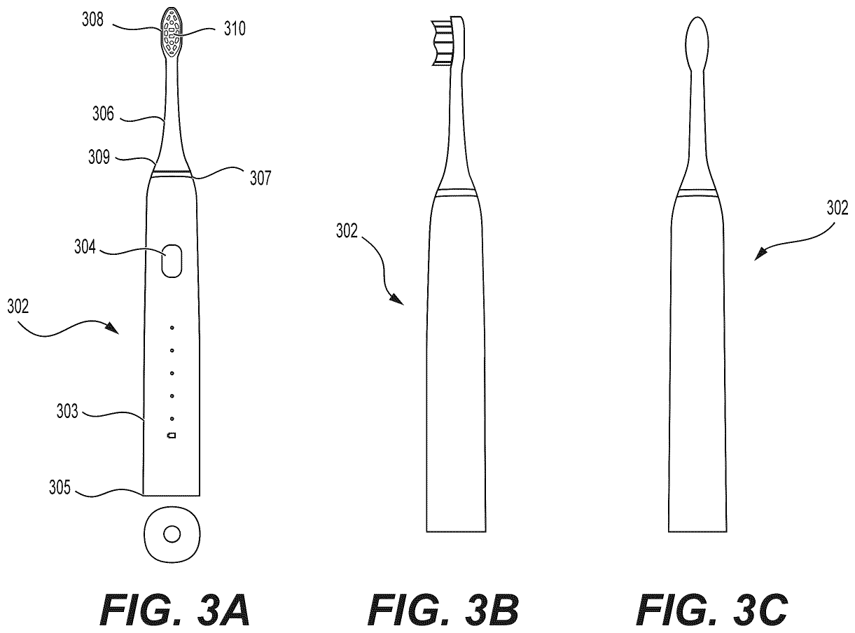

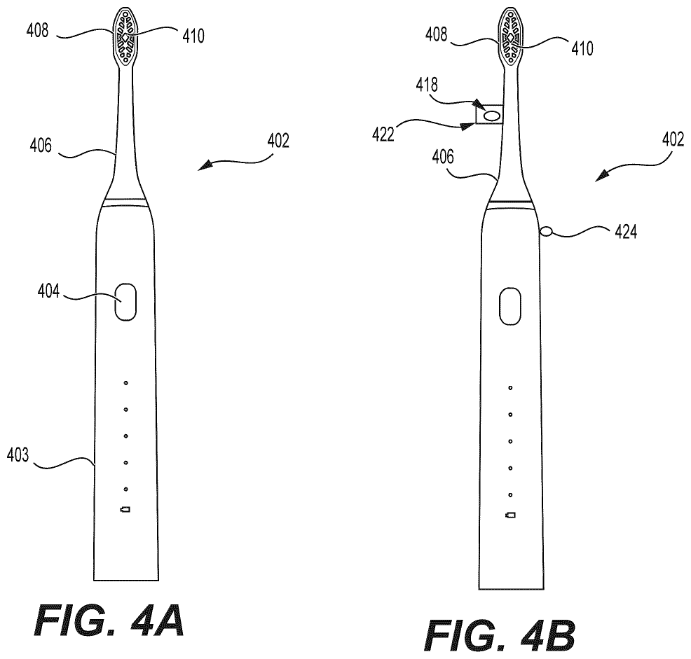

According to implementations of the disclosed subject matter, one or more light therapy oral hygiene devices (e.g., toothbrushes, as further referenced herein) with triple light emitting diodes (LEDs), are provided herein. For example, these toothbrushes may include toothbrush 102 as shown in FIG. 1A-1D, toothbrush 302, as shown in FIGS. 3A, 3B, and 3C, and toothbrush 402, as shown in FIGS. 4A and 4B. As further disclosed herein, one difference between toothbrush 102, 302, and 402 is the arrangement of the triple LEDS on each respective toothbrush. Unless specifically indicated otherwise, disclosure related to toothbrush 102, 302, and/or toothbrush 402 is provided interchangeably, such that disclosure related to one or more toothbrushes applied to each of the other toothbrushes. For example, unless indicated otherwise, handle 103 is similar to handle 303 and handle 403, input receptors 104 are similar to input receptors 304 and 404, second end 105 is similar to second end 305, detachable shaft 106 is similar to detachable shaft 306 and 406, first end 107 is similar to first end 307, head 108 is similar to head 308 and 408, first end 109 is similar to first end 309, Toothbrush 102 may be an electronic toothbrush or a manual toothbrush.

As shown in FIG. 1A and FIG. 1B, toothbrush 102 may include a detachable shaft 106 with head 108 on a first end of the detachable shaft 106. Head 108 may be angled, compact, or full size. As shown in FIG. 1A, head 108 may include a plurality of triple LEDs 110 that may be configured to selectively provide a blue light (e.g., with a wavelength of approximately 460 nm, or within a range of approximately 450 nm-495 nm), red light (e.g., with a wavelength of approximately 630 nm, or within a range of approximately 600 nm-750 nm), near infrared light (e.g., with a wavelength of approximately 850 nm, or within a range of approximately 750 nm-1000 nm) and/or a combination thereof. Generally, toothbrush 102 may be configured to provide light in the range of approximately 450 nm to approximately 1000 nm.

A plurality of LEDs 110 may be integrated into the head 108 of the toothbrush 102. The triple LEDs 110 may be manufactured or placed on strips that are inserted or placed on a printed circuit board PCB. Alternatively or in addition, the triple LEDs 110 may be manufactured as a sheet that is applied to the head 108. Alternatively, the triple LEDs 110 may be individually placed on head 108. The triple LEDs may be equidistant from each other or may be spaced such that the outside edges have a higher distribution of triple LEDs 110 and a central part has a lower distribution of triple LEDs 110, or vice versa. As shown in FIGS. 1A and 4A, triple LEDS 110 (or 410) may be arranged in a crisscross pattern, where the top left corner, middle right, and bottom right corner include triple LEDs 110 (or 410) providing blue light and the top right corner, middle left, and bottom right corner include triple LEDs 110 (or 410) providing red light. As shown in FIG. 3A, triple LEDs 310 may be arranged in two columns on head 108, where the first column includes a first triple LED providing blue light, a second triple LED providing near infrared light, and a third triple LED providing blue light and the adjacent column includes a first triple LED providing red light, a second led providing blue light, and a third led providing red light. Alternatively, the triple LEDs 110 may be arranged in any other pattern such as a circular pattern, random pattern, a design, or the like. The rows may be staggered such that the electronics for the triple LEDs 110 have sufficient space to separate from each other. In between adjacent triple LEDs 110 may be a plurality of bristles protruding from the head 108.

The detachable shaft 106 may include a metal contact (not shown) at a first end 109 of detachable shaft 106, opposite of head 108. The metal contact may be in magnetic contact with a metal contact (e.g., drive shaft 111) at a first end 107 of handle 103 to form a single piece. Accordingly, the metal contact included in detachable shaft 106 and/or the metal contact (e.g., drive shaft 111) at the first end 107 of handle 103 may be magnetic or include magnetic components. In addition, a user may be able to separate detachable shaft 106 from handle 103 to connect a different shaft with handle 103. For example, a user may replace an angled head with a compact head or full size head.

Handle 103 of toothbrush may include a recess (not shown) at a second end 105 of handle 103. The recess at second end 105 of handle 103 may be configured to communicate with charging dock 112 to charge a battery (not shown) inside handle 103, as further described herein.

The handle 103 of toothbrush 102 may be powered by via a battery (not shown) that is charged via charging dock 112. Triple LEDs 110 (shown in FIG. 1A) may be powered by via a battery (not shown) inside detachable shaft 106. A battery inside either handle 103 or detachable shaft 106 may be a power bank (e.g., configured to power an approximately 40 W, approximately 100 W, and/or approximately 200 W). A battery inside either handle 103 or detachable shaft 106 may be a lithium-ion battery. Accordingly, both the handle 103 and the detachable shaft 106 may be powered. According to an embodiment, power from the handle 103 may be transferred to the detachable shaft 106. According to another embodiment, a component (e.g., in detachable shaft 106) may be charged based on power transmitted from handle 103.

According to an alternative embodiment, a battery inside either handle 103 or detachable shaft 106 may be a water-based battery. An overcharge protection or monitoring system may be implemented to monitor electrolysis or overcharging, temperature, and charging voltage of the water-based battery. For example, when the water-based battery exceeds a water electrolysis threshold, a temperature threshold, or a charging voltage threshold, the overcharge protection or monitoring system may stop charging of the water-based battery until the amount of water electrolysis, temperature, and charging voltage are below their respective threshold.

According to an alternative embodiment, a battery inside either handle 103 or detachable shaft 106 may be a calcium-based battery. A thermal cut-off or voltage monitoring system may be implemented to monitor charging voltage, temperature, charge rate, and cycle life of the calcium-based battery. For example, when the calcium-based battery exceeds a charging voltage threshold, a temperature threshold, or a charging rate threshold, the thermal cut-off or voltage monitoring system may stop charging of the calcium-based battery until the charging voltage, temperature, and charging rate are below their respective threshold.

According to an alternative embodiment, a battery inside either handle 103 or detachable shaft 106 may be a methylene blue-based battery. A monitoring system may be implemented to monitor electrochemical stability, charging voltage, temperature, and charge rate of the methylene blue-based battery. For example, when the methylene blue-based battery exceeds a charging voltage threshold, a temperature threshold, or a charging rate threshold, the monitoring system may stop charging until the charging voltage, temperature, and charging rate are below their respective threshold.

According to an alternative embodiment, a battery inside either handle 103 or detachable shaft 106 may be a lithium-sulfur battery. A monitoring system may be implemented to monitor dendrite formation, charging voltage, temperature, and charge rate of the lithium-sulfur battery. For example, when the lithium-sulfur battery exceeds a charging voltage threshold, a temperature threshold, or a charging rate threshold, the monitoring system may stop charging of the lithium-sulfur battery until the charging voltage, temperature, and charging rate are below their respective threshold.

According to an alternative embodiment, a battery inside either handle 103 or detachable shaft 106 may be a solid-state battery. A monitoring system may be implemented to monitor dendrite formation, pre-heating, charging voltage, pressure application, and charge rate of the solid-state battery. For example, when the solid-state battery exceeds a charging voltage threshold, a pressure application threshold, or a charging rate threshold, the monitoring system may stop charging of the solid-state battery until the charging voltage, pressure application, or charging rate are below their respective threshold.

According to an alternative embodiment, a battery inside either handle 103 or detachable shaft 106 may be a sodium-ion battery. A monitoring system may be implemented to monitor stability of the anode and cathode, charging voltage, temperature, and charge rate of the sodium-ion battery. For example, when the sodium-ion battery exceeds a mechanical stress threshold, a temperature threshold, or a charging rate threshold, the monitoring system may stop charging of the sodium-ion battery until the mechanical stress, temperature, and charging rate are below their respective threshold.

According to an alternative embodiment, a battery inside either handle 103 or detachable shaft 106 may be a flexible and wearable battery. A monitoring system may be implemented to mechanical flexibility, temperature, and charge rate of the flexile and wearable battery. For example, when the flexible and wearable battery exceeds a charging voltage threshold, a temperature threshold, or a charging rate threshold, the monitoring system may stop charging of the flexible and wearable battery until the charging voltage, temperature, and charging rate are below their respective threshold.

According to an embodiment, a battery inside either handle 103 or detachable shaft 106 may be wirelessly charged when the battery is within a threshold proximity of charging dock 112 and/or handle 103, as shown in FIG. 1A. The battery may be charged using any applicable wireless technology such as near field communication (NFC), Qi technology, and/or the like. The threshold proximity may be met when, for example, toothbrush 102 is docked to charging dock 112. Alternatively, or in addition, the threshold proximity may be met when, for example, detachable shaft 106 (including the battery) is connected to handle 103.

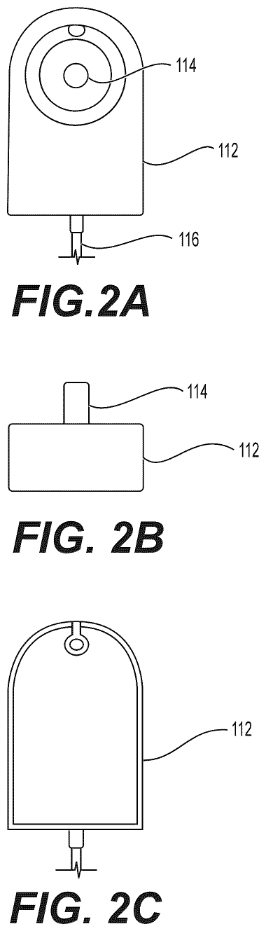

A battery in handle 103 may be charged when the recess at second end 105 of handle 103 communicates with the protrusion 114, as shown in FIG. 2A, of the charging dock 112. Moreover, as shown in FIGS. 2A, 2B, and 2C, charging dock 112 may include a connection wire 116 connected to a plug (not shown) configured to communicate with an outlet to supply power. Furthermore, as shown in FIG. 1A, when the metal contact of detachable shaft 106 is in magnetic connection with the metal contact of handle 103 and the recess of handle 103 is in communication with the protrusion 114 of charging dock 112, the charging dock is configured to supply power to charge the battery inside the detachable shaft 106. In an alternative embodiment, detachable shaft 106 may have a recess instead of a metal contact such that the detachable shaft 106 may directly communicate with protrusion 114 of charging dock 112 to supply power to the batter inside detachable shaft 106.

In embodiments, the battery in either the handle 103 or detachable shaft 106 may be charged in any applicable manner such as a Universal Serial Bus (USB) charger, wireless (e.g., Qi) charger, magnetic connection charger, adaptor, and/or connection wire, and/or the like. Handle 103 and detachable shaft 106 may include an indication of a low battery directly on handle 103 or detachable shaft 106 and/or may provide the indication via a user device (e.g., mobile phone) in connection with toothbrush 102. Accordingly, toothbrush 102 may be powered by a power connection, may be powered by a battery, and/or both.

As shown in FIG. 1C, handle 103 may include a drive shaft 111 extending from an interior of the handle 103 to an exterior of second end 105 of handle 103. The drive shaft 111 may be formed of stainless steel, titanium, or another corrosion-resistant metal capable of withstanding repeated exposure to moisture, toothpaste, saliva, and other oral-care fluids. In some embodiments, a polymeric or elastomeric coating may be applied over all or a portion of the drive shaft 111 to reduce wear, provide noise damping, or improve the seal between the handle 103 and a removable brush head.

Drive shaft 111 may be mechanically coupled to a motor assembly disposed within the handle 103. The motor assembly may include a rotary motor, a linear motor, or a piezoelectric actuator configured to impart motion to the drive shaft 111. Drive shaft 111 may rotate continuously in one direction, oscillate between clockwise and counterclockwise angular positions, or translate in a reciprocating linear fashion. In some embodiments, drive shaft 111 may operate at a high frequency (e.g., between approximately 100 Hz and approximately 500 Hz) to generate sonic or ultra-sonic vibrations for enhanced cleaning.

In some embodiments, the geometry of the drive shaft 111 may include one or more flats, keyways, splines, or other torque-transmitting features interfacing with detachable shaft 106. Such features may be dimensioned to reduce slippage, enhance torque transfer, and minimize vibration-induced wear. Drive shaft 111 may further include integrated seals, bushings, or bearings configured to inhibit fluid ingress into the handle 103 and prolong the service life of the oral device.

According to embodiments, a magnetic connection may be formed between the first end 107 of handle 103 and end 109 of detachable shaft 106. The magnetic connection may be based on magnets and/or metal components located at an edge of first end 107 of handle 103 and/or end 109 of detachable shaft 106. Alternatively or in addition, drive shaft 111 may be magnetically attached to a detachable shaft 106 component, as discussed herein.

FIG. 1D shows a cross-sectional view of toothbrush 102. As shown in FIG. 1D, when detachable shaft 106 is magnetically connected with handle 103, drive shaft 111 extends from an interior of first end 107 of handle 103 into an interior of the detachable shaft 106 through first end 109. Drive shaft 111 may transmit mechanical motion, such as rotation, oscillation, or linear reciprocation, from a motor assembly housed in the handle 103 to head 108 mounted on detachable shaft 106.

The drive shaft 111 may incorporate one or more electrical contacts 117 integrated directly or indirectly into the drive shaft 111 to provide electrical power, control signals, or both to one or more electronic components located within the detachable shaft 106 and/or head 108. As shown in FIG. 1D, the electrical contact 117 may be operably coupled via a first (e.g., positive) lead wire 119 to a main control board disposed within the handle 103. The main control board, which may be electrically connected to a rechargeable battery or other energy storage device located within the handle 103, may selectively provide electrical power to a motor assembly mechanically coupled to the drive shaft 111. The motor assembly may be configured to cause the drive shaft 111 to rotate, oscillate, or otherwise reciprocate in order to transmit mechanical motion to the brush head 108. In some embodiments, the motor assembly may include a rotary DC motor, a stepper motor, a linear actuator, or a piezoelectric transducer. Vibration frequency, torque output, and motion amplitude may be electronically adjusted via the main control board to accommodate different brushing modes, such as gentle cleaning, deep cleaning, polishing, or gum massage.

When the detachable shaft 106 is magnetically coupled to the handle 103, the main control board may further provide electrical power to electronic components disposed within the detachable shaft 106 and/or the brush head 108. For example, a lead wire 113 (e.g., a positive lead wire) disposed within the detachable shaft 106 may establish electrical connection with the electrical contact 117 integrated into the drive shaft 111, thereby completing a power circuit between the main control board and the triple LEDs 110. One or more corresponding negative or ground connections 115 may be provided via separate conductive pathways, such as an additional contact 121 disposed on the drive shaft 111, a conductive ring positioned within the magnetic coupling interface, or a chassis-ground return path formed through magnetically conductive components. In embodiments, the electrical path may include current-limiting or voltage-regulating components, such as resistors, diodes, or integrated driver circuits, to protect the triple LEDs 110 from overcurrent conditions or to implement programmable lighting patterns. An LED board located within the detachable shaft 106 may be electrically coupled to the main control board via conductive wires routed through the detachable shaft 106. The wires may be twisted, coaxial, or ribbon-type conductors, and may be encapsulated within an insulating sleeve or potting compound to increase durability and improve water resistance.

In some embodiments, the electrical contacts 117 may comprise spring-loaded pins, conductive pads, concentric conductive rings, magnetic pogo connectors, or inductive couplers that automatically establish electrical coupling upon magnetic attachment of the detachable shaft 106 to the handle 103. The contacts may be gold-plated, nickel-plated, or otherwise coated to enhance corrosion resistance and maintain reliable conductivity over repeated coupling cycles. Such an arrangement may eliminate the need for manual alignment of conductive elements, reduce manufacturing complexity, and enhance water resistance by maintaining a sealed interface. In certain designs, the contacts may be configured to break the circuit automatically when the detachable shaft 106 is removed, thereby improving user safety and preserving battery life

In some embodiments, the drive shaft 111 may further include integrated data contacts, wireless transceivers, or inductive communication elements configured to transmit control signals, such as LED activation patterns, brushing feedback, or head identification information, between the handle 103 and the detachable shaft 106. The communication may be unidirectional or bidirectional, may use digital or analog signaling, and may include error detection or encryption features. In some embodiments, multiple electrical pathways may be routed through concentric or coaxial regions of the drive shaft 111 to separate power delivery from signal communication, reducing interference and allowing for simultaneous operation of motor control, LED driving, and data transfer functions

In some embodiments, the magnetic connection between the detachable shaft 106 and the handle 103 may serve both as a mechanical retention mechanism and as part of the electrical pathway. For instance, a magnetically conductive core of the drive shaft 111 may be configured to complete a circuit when in proximity to a corresponding magnetic element in the detachable shaft 106, thereby selectively powering the LEDs 110 only when the toothbrush 102 is fully assembled. Magnets may be permanent magnets, electromagnets, or hybrid structures. In some embodiments, the magnetic field strength may be sufficient to hold the detachable shaft 106 in place during normal operation but allow easy removal when a predetermined pull force is applied, thereby balancing user convenience and operational safety. Additional sealing features, such as O-rings, gaskets, or hydrophobic coatings, may be provided at the coupling interface to prevent fluid ingress and extend the operational life of the device.

Accordingly, power and/or control signals may be transmitted from one or more components of handle 103 to one or more components of drive shaft 106 and/or head 108. For example, an LED board located at detachable shaft 106 may be powered by and/or controlled by a main board (e.g. circuit board) located at handle 103. Such control signals may be used to modify operation of the LEDs 110 such as any visible properties of LEDs 110. For example, drive shaft 111 and related components may transmit signals to LEDs 110 to synchronize or modify light outputs based on operation of the handle 103 (e.g., based on vibrations or other applicable operations discussed herein).

According to embodiments of the disclosed subject matter, power and/or control signals may be received from handle 103 components to LEDs 110 and/or circuitry connected to LEDs 110 based on magnetic powering. For example, a magnetic field may be generated between or by drive shaft 111 and one or more metal or magnetic components of detachable shaft 106. Properties of the magnetic fields (e.g., strength, power, frequency, phases, etc.) may be controlled by a main board located at handle 103, as discussed herein (e.g., using components such as lead wire 119, electrical contacts 117, etc.). The magnetic fields may induce an electric current which powers the LEDs 110 and/or related components such as an LED board. The magnetic fields may be generated in response to activation of power provided from the handle 103 to detachable shaft 106. For example, magnetic power transfer may be performed to transmit energy via electromagnetic fields generated by a transmitter coil associated with the handle 103 and a receiver coil associated with the detachable shaft 106. As a specific example, power transmitted via lead wire 119 may be magnetically transferred to a receiver component of detachable shaft 106 and carried to an LED board via electrical contacts 117. Such use of magnetic power transfers may allow for safe and efficient power transfer as compared to electronic connectors. Further, a detachable shaft having LEDs powered using such magnetic power transfer may allow for resource effective replacement of components for hygienic benefit (e.g., replacement of detachable shaft 106 which interacts with an oral cavity, and thus is more susceptible to collection of bacteria, germs, particle growth, etc.).

Handle 103 of toothbrush 102 may include one or more input receptors 104 which may be knobs, buttons, touch points (e.g., haptic response points), or the like, that may be accessible to a user. Alternatively or in addition, the input receptors may be or may include input sensors for voice and/or gesture activation. The input receptors 104 may include a power button on handle 103 to configure toothbrush 102 between an on or off state. According to implementations, toothbrush 102 may include a timer, a setting adjustor, or the like. According to an implementation, two or more tasks may be performed by the same input receptor (e.g., power and timer operation may be conducted using the same input receptor). The input receptors may be used to adjust a setting of toothbrush 102 which may include a wavelength, an intensity, a duration of activation, or the like. The settings may include treatment modalities (e.g. Clean, Sensitive, Gum Care, White, Polish, etc.) such that a user may select a preprogrammed setting and toothbrush 102 may be configured based on the selected treatment modality. The settings may configure toothbrush 102 by adjusting one or more properties such as one or more of a wavelength, intensity (e.g. may approximately be a low setting at 1 and the highest setting at 5), duration, or the like based on the setting itself (e.g., if the setting adjusts one or more of the wavelength, intensity, duration) or based on the selection of a treatment modality. As an example, toothbrush 102 may be configured using a plurality of intensity settings (e.g., up to 5 discrete settings, up to 10 discrete settings, a gradient of settings from a lowest to a highest intensity, etc.). The intensity may be adjusted by user input (e.g., via one or more input receptors, as discussed herein) and/or automatically based on an output (e.g., based on a machine learning output, as discussed herein).

The toothbrush 102 may include a timer configured to automatically shut-off the toothbrush 102 after a given amount of time. The given amount of time may be pre-determined or dynamically determined. A pre-determined amount of time may be set during manufacture of toothbrush 102 (e.g., may be 1 minute, 2 minutes, etc.) and/or may be determined based on the intensity of the light (e.g., red light, infrared light) being emitted. For example, a user may set the timer while using the toothbrush 102 at a higher light intensity setting. A user may set the timer using an input receptor and/or via the GUI of an application used to control the toothbrush 102. The timer may automatically be set for 1 minute based on the higher light intensity setting. At a different time, the user may set the timer while using the toothbrush 102 at a lower light intensity setting relative to the higher intensity setting. The timer may automatically be set for 2 minutes based on the lower light intensity setting.

The irradiance of the toothbrush 102 may be between approximately 5 mW/cm² and approximately 150 mW/cm². For example, the irradiance of the toothbrush 102 may be approximately 7 mW/cm². The irradiance may be variable based on one or more settings (e.g., as output by a machine learning model, set by a user, etc.).

According to implementations of the disclosed subject matter, the toothbrush 102 may emit less than 1 V/m at less than 0.5 inches away or less. For example, the toothbrush 102 may emit 0 V/m at less than 0.5 inches away. Such emission may be considered a safe amount of emission for use of the toothbrush 102.

According to implementations of the disclosed subject matter, the triple LEDs 110 of toothbrush 102 may fluctuate at a frequency of 3 Hz or less. The fluctuation may be at a level that a human eye cannot perceive flickering of the triple LEDs 110 of toothbrush 102.

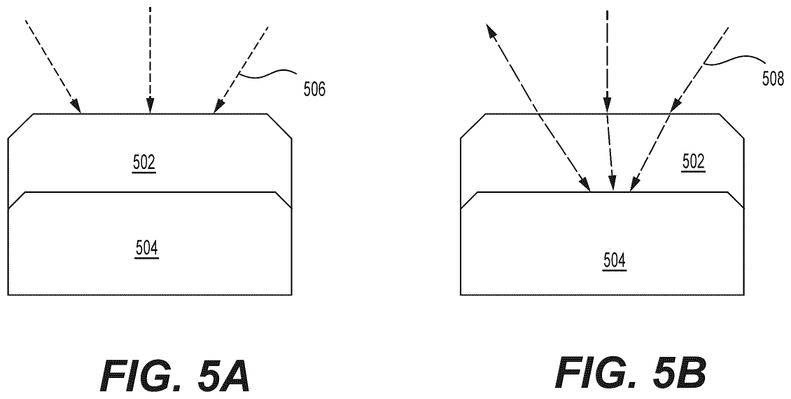

The triple LEDs 110 disclosed herein may be each be configured to emit light (e.g., red light or blue light) that can interact with cells on a surface as well as light (e.g., near infrared light) that can interact with cells deeper than a surface. Such a configuration has added health benefits in comparison to a configuration that only emits light incident on, for example, a surface level. FIGS. 5A and 5B depict diagrams representing an oral cavity's upper surface 502 (e.g., a gum surface) and a sub-surface area 504 (e.g., gum tissue). As disclosed herein, the toothbrush 102 may include triple LED circuits each configured to emit red light, near infrared light, blue light, and/or a combination of the three. The ability to emit red light, near infrared light, blue light and/or a combination of the three may result in the toothbrush 102 efficiently reducing inflammation, increasing circulation, and/or optimizing functionality of mitochondria. As shown in FIG. 5A, red light or blue light 506 may emit from the toothbrush 102 and be incident on the upper surface 502. The red light or blue light 506 may interact with the upper surface 502 to reduce inflammation, increase circulation, and/or optimize functionality of mitochondria, or the like, at the upper surface 502. Additionally, as shown in FIG. 5B, near infrared light 508 may interact with the sub-surface area 504 to reduce inflammation, increase circulation, and/or optimize functionality of mitochondria, or the like, at the sub-surface area 504. Accordingly, using the triple LED circuits, the toothbrush 102 may have an effect on both the surface and sub-surface tissue that it interacts with.

According to implementations of the disclosed subject matter, light therapy oral hygiene device may include one or more sensors. FIG. 4B depicts sensors 418 and 424 associated with a light therapy oral hygiene device. As shown in FIG. 4B, sensor 418 may be placed on an exterior portion of detachable shaft 406. Sensor 418 may be attached to or contained within the interior of detachable shaft 406 or handle 403. Sensor 424 may be attached to an exterior portion of handle 403.

It will be understood that are example sensors and that one or more additional sensors may be associated with light therapy oral hygiene device. Alternatively, not all the sensors shown in FIG. 4B may be included in a given light therapy oral hygiene device. Alternatively, or in addition, one or more sensors associated with the light therapy oral hygiene device may be external to the light therapy oral hygiene device. Such sensors may communicate with light therapy oral hygiene device or one or more processors associated with the light therapy oral hygiene device via a wired or wireless connection (e.g., a network connection, a Bluetooth®, connection, a near field connection, a line of sight connection, etc.). A given sensor, as those discussed herein, may be positioned, calibrated, and/or powered based on the sensing functionality of the respective sensor. For example, sensors configured to detect tissue properties may be positioned on the interior portion of light therapy oral hygiene device and sensors configured to detect saliva properties may be positioned on the exterior portion of light therapy oral hygiene device.

The light therapy oral hygiene device may be used to whiten teeth, promote oral health, and/or treat or mitigate oral health conditions such as inflammatory conditions, gingivitis, circulation, oral mycosis, or the like. The light therapy oral hygiene device may also be used to expedite recovery (and reduce inflammation and/or pain) after oral surgery or other dental procedures. The light therapy oral hygiene device based on the use of both the red light, blue light, and the near infrared light, may reduce inflammation, kill bacteria, increase circulation, and optimize the functionality of mitochondria (e.g., to allow cells to generate energy more efficiently). As further discussed herein, the combination of the red light, blue light, and near infrared light may be used to treat both surface conditions and sub-surface conditions (e.g., at a tissue level).

According to an implementation of the disclosed subject matter, light therapy oral hygiene device may include a sensor to detect an oral condition. The oral condition may be any applicable condition including, but not limited to, inflammatory conditions, gingivitis, circulation, oral mycosis, or the like. The sensor may be a visual sensor, ambient condition detection sensor, pH sensor, biochemical sensor, or the like. The light therapy oral hygiene device may be configured to adjust a setting based on the data obtained by the sensor. For example, the sensor may collect data and provide the data to a machine learning model. The machine learning model may be trained to output one or more settings of the toothbrush 102 based on the input sensor data. The output may be based on detecting a given oral condition or may be based on an oral condition known to or provided to the machine learning model. For example, the sensor data may indicate a given oral condition and the machine learning model may output one or more parameters to operate the light therapy oral hygiene device in order to treat and/or mitigate the expansion of the oral condition. Alternatively, the machine learning model may be a clinical decision support engine that may receive oral information from clinical guidelines, a user's dentist (e.g., via a server, network, or other connection to the user's dental data). Based on the user's oral information, the machine learning model may output parameters for operation of the light therapy oral hygiene device to treat or mitigate any conditions included in the oral information.

The output may include a wavelength or set of wavelengths to output using the light therapy oral hygiene device. The output may also include duration data such that the timer can be dynamically set to turn the light therapy oral hygiene device off after the duration of time output by the machine learning model. Alternatively, the timer may be dynamically set to change a wavelength after a duration of time output by the machine learning model.

According to an implementation of the disclosed subject matter, settings for the light therapy oral hygiene device may be adjusted based on user or automated input. As disclosed above, a machine learning model may output settings adjustments (e.g., intensity, wavelength, duration, etc.) based on sensor data and/or data received at the machine learning model. One or more settings of the light therapy oral hygiene device may be adjusted based on user input. A user may provide the user input directly via input recipients on the light therapy oral hygiene device. Alternatively, the light therapy oral hygiene device may be connected to a user device (e.g., via a network, wired, or other wireless connection). The user may connect to the light therapy oral hygiene device via an application (e.g., a mobile device application, a website or web application, a standalone controller, etc.) and provide setting input via a graphical user interface (GUI) of the application.

According to implementations of the disclosed subject matter, the light therapy oral hygiene device may incorporate security features. For example, a sensor may detect when the light therapy oral hygiene device is placed inside an oral cavity (e.g., a pressure sensor to measure cheek/lip/teeth pressure, a moisture sensor, a biochemical sensor, etc.). Accordingly, the light therapy oral hygiene device may activate only when the sensor determines that the light therapy oral hygiene device is inside the oral cavity. Alternatively, or in addition, the light therapy oral hygiene device may include a position or orientation sensor such that the light therapy oral hygiene device activates only when the position or orientation sensor detects that the light therapy oral hygiene device is in a correct position and/or orientation. For example, the position or orientation sensor may deactivate the light therapy oral hygiene device when it is removed from a user's oral cavity to prevent the red, blue, and/or near infrared light from being incident up on the user's eyes. The light therapy oral hygiene device may also include a time used detection mechanism to detect how long a user has used the toothbrush 102 and/or at what intensity the light therapy oral hygiene device was used for the duration. If the time used detection mechanism determines that the light therapy oral hygiene device was used for a duration greater than a recommended duration and/or at an intensity greater than a recommended intensity for a given duration, the time used detection mechanism may automatically shut off the light therapy oral hygiene device or reduce the intensity of the light therapy oral hygiene device.

The sensors shown in FIG. 4B or otherwise discussed herein may be a visual sensor, chips, laser sensor, temperature sensors, ambient condition detection sensor, pH sensor, biochemical sensor, motion sensors, material sensors, or the like. The sensors shown in FIG. 4B or otherwise discussed herein may be configured to sense oral sensed data which may include, but is not limited to, data related to biometric information, exhaled breath condensate (EBC), PH levels, saliva, chemicals, shapes, objects, food or food particles, electrical signals, tooth data, mitochondria, proteins, glucose, lactate, urea, uric acid, microorganisms, serum, blood, and/or the like. The oral sensed data may be used to detect an oral condition or a treatment.

The sensors shown in FIG. 4B or otherwise discussed herein may be powered using the same power source (e.g., a battery) as the light therapy oral hygiene device. Alternatively, or in addition, the sensors may be powered using an external energy source (e.g., a power source, kinetic energy, heat energy, etc.).

The sensors shown in FIG. 4B or otherwise discussed herein may be activated continuously or based on one or more triggers. A sensor activation may correspond to sensing of given sensed data by a given sensor. A sensor activation may include receiving an input (e.g., a physical input, a biochemical input, an electrical input, a motion input, etc.) and/or may include converting an input to a sensed signal. The sensed signal may be an electrical signal, a change of a state, a change of a property, or the like. For example, a sensor may be configured to detect pH levels. The sensor may continuously sense pH levels such that a pH detection mechanism constantly receives a sample (e.g., a saliva sample), applies the sample to a pH detection strip, and generates a signal (e.g., a reading) based on a physical property change of the strip.

A triggered based sensor activation may be based on a temporal trigger (e.g., based on a time, a duration of time, a chronic time, etc.), or an event based trigger. An event based trigger may be a trigger that is activated upon the occurrence of a given event. An event based trigger may be based on a signal from a sensor. For example, a position sensor or group of sensors may determine that the light therapy oral hygiene device is in a user's mouth based on meeting one or more pressure criteria. The pressure sensor or group of sensors may detect that their proximity to corresponding areas of an oral cavity meets one or more thresholds. Accordingly, the pressure sensor or group of sensors may emit a signal indicating that the light therapy oral hygiene device is in a user's mouth. Based on the signal, one or more other sensors may be activated, where the event in this case is the pressure sensor or group of sensors emitting the signal indicating that the light therapy oral hygiene device is in a user's mouth.

An event based trigger may be based on an output determined based on user information, sensed information, or the like. User information may be a user history, a current user state, or the like. A current user state may be received from one or more components such as external sensors, a database, or the like (e.g., a blood pressure device, a health care database, etc.). The user information and/or sensed information (e.g., sensed by one or more sensors associated with the light therapy oral hygiene device) may be input at a machine learning model and the machine learning model may determine when to trigger a sensor activation based on the user information and/or sensed information.

According to an implementation of the disclosed subject matter, one or more sensors associated with a light therapy oral hygiene device may sense biochemical markers in bio fluids, such as sweat, tears, saliva and/or interstitial fluid. Such sensors may be non-invasive and may include one or more electrochemical and/or optical biosensors. Data sensed using such sensors may be used to identify or determine information related biomarkers including metabolites, bacteria, and hormones. For example, as shown in FIG. 4B, saliva may be collected within saliva collector 422 of toothbrush 402. The saliva may be in contact with a non-invasive electrochemical sensor such as sensor 418 configured to detect one or more biomarkers including bacteria from the saliva. Concentration of certain biochemical markers in saliva may be highly relevant to those in blood, as a result of exchange between salivary glands and blood. Accordingly, as further discussed herein, sensed data (e.g., sensed salvia) may be used to determine or predict blood properties.

The non-invasive electrochemical sensor may sense electrochemical attributes of the saliva and may generate electrical signals based on the same. The electrical signals may be received at a processor located at or remote from the light therapy oral hygiene device. The processor may convert the electrical signals to data that represents the presence of one or more biomarkers (e.g., a type, quantity, quality, etc., of bacteria). Alternatively, the non-invasive electrochemical sensor may itself be configured to output such data.

According to an implementation of the disclosed subject matter, one or more sensors associated with a light therapy oral hygiene device may sense EBC content. EBC content may include, but is not limited to, mediators including adenosine, ammonia, hydrogen peroxide, isoprostanes, leukotrienes, nitrogen oxides, peptides and cytokines. Concentrations of these mediators are influenced by lung diseases and modulated by therapeutic interventions. Similarly, such one or more sensors may detect PH levels based on collected EBC. As further discussed herein, properties of EBC content and/or changes in the same may indicate the presence or probability of conditions (e.g., respiratory conditions).

According to an implementation of the disclosed subject matter, one or more sensors associated with a light therapy oral hygiene device may sense volatile organic compounds (VOCs) biomarkers. VOC biomarkers may be indicative of environmental exposures such as that caused by particulate matter from burn pits, oil field fires, metal alloys, or the like.

According to an implementation of the disclosed subject matter, one or more sensors associated with a light therapy oral hygiene device may be laser sensors. Such sensors may use one or more lasers to detect oral cavity properties. For example, laser sensors and the corresponding sensed data may be used for the early detection of decay. The laser sensors may be used to detect lesions (e.g., in early stages). A laser sensor may be placed on or near one or more teeth via the light therapy oral hygiene device. The laser sensors may generate a digital readout, which may be used to determine tooth decay.

According to an implementation of the disclosed subject matter, one or more sensors associated with a light therapy oral hygiene device may be configured to detect dental caries. Dental caries may be an infection resulting from tooth-adherent cariogenic bacteria, primarily Streptococcus Mutans, which metabolize sugars to produce acid, demineralizing the tooth structure over time. The sensors and/or a component of the light therapy oral hygiene device may be configured to sense such bacteria, sugar, and/or acid and may include fluorescent components to facilitate detection of dental caries. The fluorescent components may include zinc oxide quantum dots/poly(dimethylsiloxane) (ZnO/PDMS) nanocomposite.

According to an implementation of the disclosed subject matter, one or more sensors associated with a light therapy oral hygiene device may be pressure sensors, force sensors, or tooth movement sensors for orthodontic treatment monitoring. Such sensors may measure the force applied by orthodontic devices and/or any tooth changes based on the same. Such sensors may capture changes in tooth position over time. Such sensors may be implemented using a bracket or other component configured to detect force-moment (e.g., six force moment) of wires and/or brackets at one or more teeth. One or more stress sensors may be integrated on a chip via complementary metal oxide semiconductor (CMOS) technology. The chip may embedded into the light therapy oral hygiene device. Based on the measured data, force-moment detection be determined. The data may be applied to or using one or more simulations. For example, isolated calibration loads may be complemented by using finite element (FE) simulations.

According to an implementation of the disclosed subject matter, one or more sensors associated with a light therapy oral hygiene device may be temperature sensors. The temperature sensors may be used, for example, for monitoring peri-implant diseases. Temperature may be a targeting parameter of inflammation and the local temperature near a tooth or implant may be used as an indicator to monitor peri-implant diseases. Accordingly, a multi-channel temperature sensors may be microfabricated based on a photo-definable polyimide. Such sensors may output temperatures (e.g., over time) to detect temperature changes and/or peri-implant diseases.

According to an implementation of the disclosed subject matter, one or more sensors associated with a light therapy oral hygiene device may be chemical sensors. Such sensors may sense food intake. Such sensors may include soft, low-profile, intraoral electronics configured for continuous real-time monitoring of sodium ingestion. Such sensors may include sodium ion-selective sodium electrodes (ISE), made of polymers with high selectivity, wide signal range, and rapid response time, selected for monitoring sodium levels in saliva.

According to an implementation of the disclosed subject matter, one or more sensors associated with a light therapy oral hygiene device may be food physical sensors. Physical sensors may capture the motion of oral activity. The oral activity may be sensed by such sensors and may be provided to a processor, as discussed herein. The oral activity may be provided as an input to a machine learning model which may generate a machine learning output, as further discussed herein. The machine learning output may be based on comparing the oral activity to known activity associated with known conditions (e.g., grinding) to identify an oral condition.

According to an implementation of the disclosed subject matter, one or more sensors associated with a light therapy oral hygiene device may be optical sensors. Such sensors may use light-based techniques to detect oral cavity properties such as saliva properties, chemical properties, blood or blood flow properties, or the like. Such sensors may use a light-based techniques to quantify magnetic fields produced by neurons firing in the brain and may be used instead of magnetic resonance imaging (MRI) machines to create similar imaging, eliminating. Additionally, according to an implementation, the light therapy oral hygiene device may include components (e.g., copper, galvanized steel, aluminum, etc.) that provide expensive cooling or electromagnetic shielding required when undergoes an MRI scan.

According to an implementation of the disclosed subject matter, one or more sensors associated with a light therapy oral hygiene device may be infra-red or red sensors. Such sensors may capture high signal-to-noise and high-resolution photoplethysmography (PPG) measurements from deep beneath the oral cavity (e.g., up to approximately 20 mm or up to approximately 10× deeper than green light) to extract biometric sensed data.

According to an implementation of the disclosed subject matter, one or more sensors associated with a light therapy oral hygiene device may be ultraviolet (UV) sensors. The UV sensors may use UV light to generate sensed data. According to an implementation, a user may swirl a fluorescent solution in the user's mouth. The user may then insert the light therapy oral hygiene device and the UV sensors may detect oral properties (e.g., plaque) based on reminisce of the fluorescent solution. It will be understood that the fluorescent solution may not be required for the UV sensors to detect oral properties, though such a solution may improve the detectability of the same.

According to an implementation of the disclosed subject matter, one or more sensors associated with a light therapy oral hygiene device may be biosensors. Biosensors may be used to sense sensed data that allows assessment of health and disease states. The biosensors may generate signals based on oral fluids, cells, microorganisms, etc., as well as other compounds that may be found in or pass through the oral cavity.

According to an implementation of the disclosed subject matter, one or more sensors associated with a light therapy oral hygiene device may be glucose sensors. Such sensors may provide continuous glucose monitoring (CGM) based on oral cavity properties such as saliva properties. Such sensors may be configured to detect blood glucose or sense markers indicative of blood glucose. Such sensors may be configured to detect ketones and/or ketone properties which may be used to determine glucose levels. According to an implementation, such sensors may sense blood (e.g., blood that may be present in the oral cavity after brushing or flossing) and may detect blood glucose based on the blood.

According to an implementation of the disclosed subject matter, one or more sensors associated with a light therapy oral hygiene device may be mitochondrial sensors. Mitochondrial sensors may be configured for quantum sensing such as by using one or more quantum objects. A quantum object may be the unpaired electron associated with an nitrogen-vacancy (NV) center in diamond which can be exploited as an extraordinarily sensitive room temperature magnetometer, deployed for nanoscale temperature measurements. Such objects may be used to detect temperature changes, as discussed herein.

Mitochondrial sensors may include a PINK1 sensor. PINK1 is a serine/threonine protein kinase which localizes to mitochondrion and regulates its function and turnover by sensing when mitochondria are damaged. PINK1 may be used for mitochondrial health by facilitating fusion and fission, mitophagy, and mitochondrial transport pathways, which serve as a quality control system to remove dysfunctional or damaged mitochondrion from the cell. Accordingly, mitochondrial sensors may detect the presence, quality, and/or quantity of PINK1.

Mitochondrial sensors may be configured to detect fluorescence-based assays including measurements of mitochondrial calcium, superoxide, mitochondrial permeability transition, and membrane potential.

According to an implementation of the disclosed subject matter, one or more sensors associated with a light therapy oral hygiene device may be used to mimic blood tests. Such an implementation may use one or more of short-wavelength infra-red sensors, semiconductor photonics and/or electrooptic sensors, laser printed graphene (LIG) based electrode biosensors, or the like. A short-wavelength infra-red sensor may be used to detect the amount of sugar in a user's blood. A semiconductor photonics and/or electrooptic sensor may be configured to detect levels of glucose, lactate, urea, serum albumin, and/or other substances in a user's blood. LIG sensors may combine high electrical conductivity of graphene with an ultra-easy fabrication procedure that simply requires a CO2 laser printer. LIG sensors may be implemented a high porosity and an interlocking design to enhance the biosensor's sensitivity. Data output by such sensors may be used to generate results similar to a blood test.

According to implementations of the disclosed subject matter, a light therapy oral hygiene device may be configured to generate sensed data using one or more of the sensors disclosed herein. The sensed data may be processed by a processor (e.g., an internal or external processor). A machine learning model may be used to generate a machine learning output. The machine learning output may include an indication, signal, instruction, or the like to configure the light therapy oral hygiene device to output light at a wavelength, intensity, rate, duration, and/or frequency. The configuration of the light therapy oral hygiene device may be, for example, to treat or otherwise improve a condition indicated based on the sensed data.

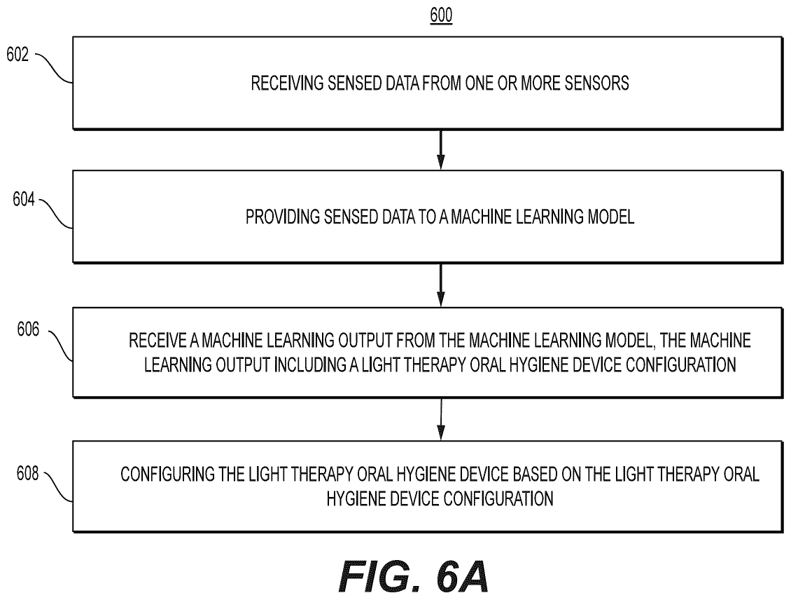

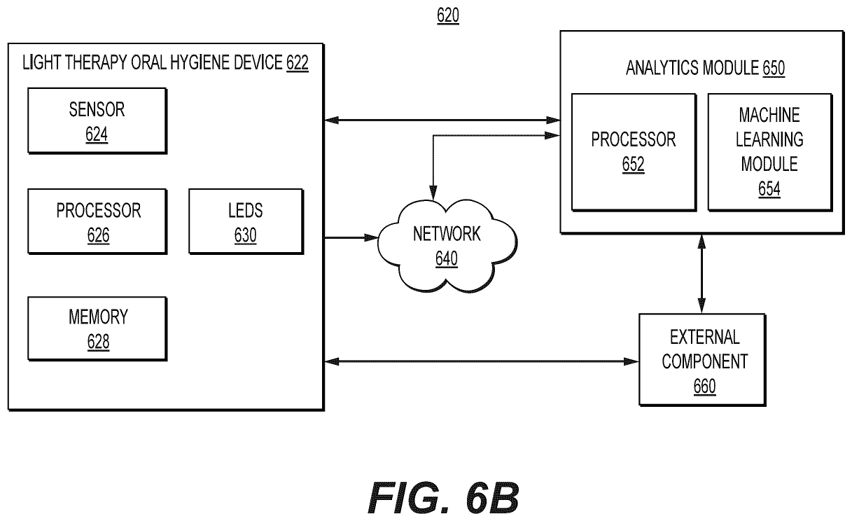

FIG. 6A depicts a flow chart 600 for targeted light therapy. At 602, sensed data from one or more sensors of the light therapy oral hygiene device may be received. The sensed data may be any of the sensed data discussed herein generated by one or more of the sensors discussed herein. The sensed data may be generated by the one or more sensors continuously or based on an event, as discussed herein. The sensed data may be received an internal or external processor, as further discussed herein in reference to FIG. 6B. The sensed data may be sensed by one or more sensors in a first format (e.g., a sensor specific format, a signal, or the like) and may be converted into a second format (e.g., by a processor).

At 604, the sensed data may be provided to a machine learning model. The machine learning model may be trained based to generate a machine learning output based on sensed data. The machine learning model may be trained based on medical conditions, historical changes that effect medical conditions, light properties, or the like. The machine learning model may be trained by adjusting one or more of weights, layers, biases, nodes, or the like to correlate sensed data to medical conditions such that the correlation may be a probability or likelihood (e.g., above a respective threshold) that sensed data indicates the presence or likelihood of a medical condition. For example, the machine learning model may receive the sensed data and may apply the sensed data to one or more of weights, layers, biases, nodes, or the like to determine if the sensed data indicates the presence or likelihood of plaque. Alternatively, for example, the machine learning model may determine health properties of a user based on the sensed data. The health properties may be indicative of one or more medical conditions. The machine learning model may generate a machine learning output based on the correlation.

According to implementations of the disclosed subject matter, the machine learning model may be a single machine learning model or may include multiple machine learning models. For example, sensed data from different sensors may be provided to respective machine learning models and a central machine learning model configured to receive outputs from each of a plurality of respective machine learning models may generate the machine learning output.

According to implementations of the disclosed subject matter, the machine learning model may be trained and/or updated based on cohort data. Cohort data may correspond to sensed data and/or outcomes for a cohort of users. The cohort of users may be any group of users (e.g., other users that use similar the light therapy oral hygiene devices, users with medical conditions, users that received light therapy for medical conditions, results of light therapy for users, or the like). For example, cohort data may include actual or simulated sensed data for the cohort of users. The cohort data may further include light therapy or other treatments implemented for the cohort of users and what effect the light therapy or other treatments had for the cohort of users. Light therapy treatments or other treatments correlated to light therapy treatments that had a positive effect for the cohort of users may be weighted heavily when training the machine learning model to generate a machine learning output. Accordingly, for example, sensed data for a given user may be compared to sensed data for the cohort of users. The machine learning model may apply greater weight to the layers, biases, weights, nodes, etc. trained based on the cohort of users that match the sensed data for a given user based on a matching threshold. Further, the machine learning model may apply greater weight to light therapy that had a positive effect for those matched cohort of users, when generating a machine learning output. Positive outcomes may include a reduction in presence or intensity of a given medical condition, the treatment of a medical condition, the prevention of a medical condition, or the like, for one or more medical conditions indicated by the sensed data.

At 606, a machine learning output may be received from the machine learning model. The machine learning output may include a light therapy oral hygiene device configuration. Accordingly, the machine learning output may include a configuration that may be best indicated by the machine learning model to treat, mitigate, and/or prevent a medical condition for a given user.

A the light therapy oral hygiene device configuration may include one or more parameters of wavelengths of light, intensity of light, rate (e.g., pulse rate), duration of treatment, frequency of treatment, or the like. The wavelength(s) of light may correspond to the wavelengths that the light therapy oral hygiene device is configured to output based on one or more other parameters. The machine learning output configuration may indicate how the light therapy oral hygiene device outputs one or different wavelengths at different intensities, a given rate or different rates of output of the wavelengths, a duration or durations of time for output of the wavelengths, frequencies of output of the wavelengths, or the like. For example, the configuration may indicate that a first wavelength should be output at a first intensity for five minutes, at a second intensity for seven minutes. The configuration may further indicate that after the twelve total minutes of outputting the first wavelength, a four minute break where no wavelength is output should be implemented. The configuration may further indicate that after the break, a second wavelength should be output at a third intensity for two minutes, at a second intensity for nine minutes. The configuration may further indicate that the previous steps should be cycled through four times before the light therapy oral hygiene device automatically shuts off.

According to an implementation, sensor data from a first sensor or group of sensors may be used to generate a machine learning output. Subsequently, sensed data from a second sensor or group of sensors may be used to update the machine learning output. The sensed data from the second sensor or group of sensors may be generated based on a first machine learning output configuration indicating a request for sensor data from the second sensor or group of sensors. For example, the machine learning model may determine that sensed data from a first sensor or group of sensors is not sufficient (e.g., in quantity, quality, type of data, etc.). Accordingly, the machine learning output may indicate a request for additional data from the second sensor or group of sensors. According to an implementation, the second sensor or group of sensors may include the first sensor or group of sensors (e.g., if additional data from the same sensors is requested).

At 608, the light therapy oral hygiene device may be configured based on the light therapy oral hygiene device configuration indicated the machine learning output. The light therapy oral hygiene device may be configured using a processor, as further discussed herein. The configuration indicated by the machine learning output may be implemented until an updated configuration is received, or until the light therapy oral hygiene device is reset using a reset signal (e.g., provided by a processor or via user input).

The intensity of light output by the light therapy oral hygiene device may be adjusted by adjusting the power provided to one or more LEDs. Alternatively, or in addition, the intensity may be adjusted by a signal configured to increase or decrease the intensity output by one or more LEDs.

The wavelength output by the light therapy oral hygiene device may be adjusted by activating and/or deactivating one or more LEDs. Alternatively, or in addition, the wavelength may be adjusted by modifying a property of the one or more LEDs. For example, each LED may have an on-board chip configured to modify the wavelength output by a given LED. A triple LED may include multiple bulbs configured to output one or more wavelengths and a wavelength may be adjusted by activating respective bulbs by providing a single to the on-board chip.

According to an implementation of the disclosed subject matter, updated sensed data may be provided to the machine learning model. The machine learning model may generate an updated machine learning output based on the updated sensed data. The updated machine learning output may be adjusted based on a current configuration (e.g., a previous machine learning output). The updated machine learning output may be adjusted based on updated cohort data that may also be received at the machine learning model. Accordingly, the light therapy oral hygiene device may be continuously configured, at least in part based on changes effected by light therapy from pervious configurations and/or changes that a given user undergoes (e.g., health changes, diet changes, medication changes, etc.).

According to an implementation, the machine learning model may receive external input. The external input may be from one or more sensors external to the light therapy oral hygiene device and/or user data. User data may be user diet information, user medication information, user health information, user activity information, or the like. For example, user diet information may be input by a user or an automated system. The user diet information may be used to determine the light therapy oral hygiene device configuration such that, for example, a change to a salty diet may require changing a light (e.g., wavelength) output by the light therapy oral hygiene device.

According to an implementation, the machine learning output may include an external device configuration. For example, the machine learning model may detect a glucose level of a given user. The glucose level may indicate the requirement of additional insulin at a given time. Accordingly, the machine learning model output may include a configuration for an insulin delivery device and the output may be provided to the insulin delivery device. The insulin delivery device may adjust an insulin output based on the machine learning output.

FIG. 6B depicts a system environment 620 for targeted light therapy in accordance with the subject matter disclosed herein. A light therapy oral hygiene device 622 may include one or more sensors 624, processor 626, memory 628, and triple LEDs 630. In some implementations, processors 626 may include one or more microprocessors, microchips, or application-specific integrated circuits. Memory 628 may include one or more types of random-access memory (RAM), read-only memory (ROM), and cache memory employed during execution of program instructions and may further include storage including one or more databases, cloud components, servers, or the like. The storage may include a computer-readable, non-volatile hardware storage device that stores information and program instructions. Processor 626 may use data buses to communicate with memory 628, sensors 624, and/or triple LEDs 630. Processor 626 and/or another component (e.g., a transmitter and/or receiver) associated with the light therapy oral hygiene device 622 may be configured to transmit and/or receive data (e.g., sensed data, one or more configurations, etc.)

Analytics module 650 may be housed at the light therapy oral hygiene device 622 or may be an external component, as shown in FIG. 6B. Analytics module 650 may include a processor 652 and a machine learning module 654. Machine learning module 654 may be a set of instructions, code, or the like that are implemented (e.g., compiled) using processor 652). An external analytics module 650 may communicate with the light therapy oral hygiene device 622 via wired or wireless connection. The wireless connection may be via a network 640 such that analytics module 650 may be a remote or cloud component.

External component 660 may be an external sensor or an external device configured to communicate with the light therapy oral hygiene device 622 and/or analytics module 650 via wired or wireless connection.

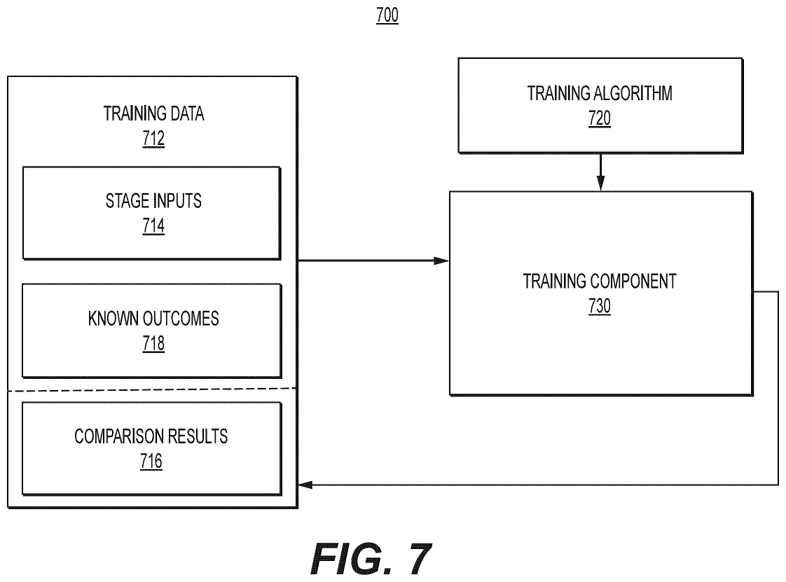

One or more implementations disclosed herein include a machine learning model. For example, as disclosed herein, a machine learning model may output operational parameters or settings to operate the light therapy oral hygiene device based on, for example sensor data regarding oral health. A machine learning model disclosed herein may be trained using the data flow 700 of FIG. 7. As shown in FIG. 7, training data 712 may include one or more of stage inputs 714 and known outcomes 718 related to a machine learning model to be trained. The stage inputs 714 may be from any applicable source including sensor data. The known outcomes 718 may be included for machine learning models generated based on supervised or semi-supervised training. An unsupervised machine learning model may not be trained using known outcomes 718. Known outcomes 718 may include known or desired outputs for future inputs similar to or in the same category as stage inputs 714 that do not have corresponding known outputs.

The training data 712 and a training algorithm 720 may be provided to a training component 730 that may apply the training data 712 to the training algorithm 720 to generate a machine learning model. According to an implementation, the training component 730 may be provided comparison results 716 that compare a previous output of the corresponding machine learning model to apply the previous result to re-train the machine learning model. The comparison results 716 may be used by the training component 730 to update the corresponding machine learning model. The training algorithm 720 may utilize machine learning networks and/or models including, but not limited to, a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as communicating with an application via a GUI, adjusting the light therapy oral hygiene device parameters or settings, etc., may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

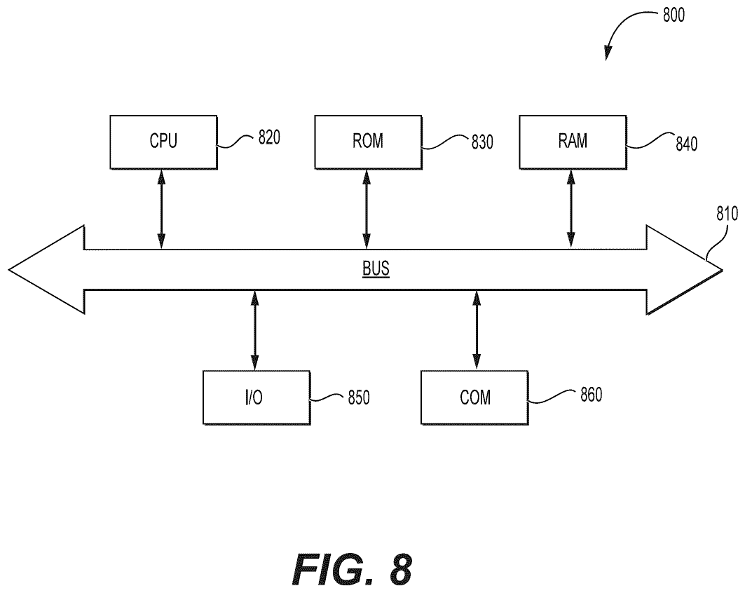

FIG. 8 depicts an example system 800 that may execute techniques presented herein. FIG. 8 is a simplified functional block diagram of a computer that may be configured to execute techniques described herein, according to exemplary embodiments of the present disclosure. Specifically, the computer (or “platform” as it may not be a single physical computer infrastructure) may include a data communication interface 860 for packet data communication. The platform may also include a central processing unit (“CPU”) 820, in the form of one or more processors, for executing program instructions. The platform may include an internal communication bus 810, and the platform may also include a program storage and/or a data storage for various data files to be processed and/or communicated by the platform such as ROM 830 and RAM 840, although the system 800 may receive programming and data via network communications. The system 800 also may include input and output ports 850 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed:

1. An oral device comprising:
a base; and
a shaft including a head with one or more triple light emitting diodes (LEDs), wherein the shaft is magnetically coupled to the base to receive magnetically transferred power;
wherein when the shaft is in magnetic contact with the base to receive the magnetically transferred power, the one or more triple light emitting diodes (LEDs) are configured to emit light.

2. The oral device of claim 1, further comprising:
a drive shaft extending from an interior of the base to an interior of the shaft.

3. The oral device of claim 2, wherein the drive shaft includes an electrical contact, the electrical contact is electrically coupled to a power source to provide power to the drive shaft.

4. The oral device of claim 3, wherein the electrical contact is electrically coupled to the one or more triple LEDs to provide power to the one or more triple LEDs.

5. The oral device of claim 3, wherein the electrical contact is electrically coupled via a lead wire to a main control board disposed within the base.

6. The oral device of claim 5, wherein the main control board is electrically coupled to the power source disposed within the base and configured to provide electrical power to a motor assembly mechanically coupled to the drive shaft.

7. The oral device of claim 6, wherein the motor assembly is configured to cause the drive shaft to oscillate and transmit mechanical motion to the head.

8. The oral device of claim 1, wherein each triple LED is configured to output a first light having a first wavelength, a second light having a second wavelength, and a third light a third wavelength.

9. The oral device of claim 8, wherein the first light is a blue light, the first wavelength approximately 450 nm-495 nm, the second light is a red light, the second wavelength is approximately in a range of 600 nm-750 nm, the third light is a near red light, the second wavelength is approximately in a range of 750 nm-1000 nm.

10. The oral device of claim 1, further comprising:
a processor, wherein the processor is configured to:
output sensed data sensed by one or more sensors;
receive an oral device configuration based on the sensed data, the oral device configuration comprising one or more of wavelengths of light, intensities of light, rates, durations, or frequencies for configuring the oral device; and
configure the oral device based on the oral device configuration.

11. The oral device of claim 10, wherein the oral device configuration is generated by a machine learning model based on the sensed data.

12. The oral device of claim 1, wherein the base includes an input receptor, wherein the input receptor comprises a power control configured to control the one or more of wavelengths of light, intensities of light, rates, durations, or frequencies of the one or more triple LEDs.

13. A system for providing light therapy to an oral cavity, the system comprising:
an oral device comprising:
a base;
a shaft magnetically coupled to the base to receive magnetically transferred power;
one or more sensors attached to the base or shaft;
one or more triple light emitting diodes (LEDs) disposed on a head of the shaft, wherein each triple LED is configured to output red light, near red light, and blue light;
at least one memory storing instructions; and
at least one processor executing the instructions to perform a process, the processor configured to:
receive sensed data sensed by the one or more sensors;
receive an oral device configuration based on the sensed data, the oral device configuration comprising one or more of wavelengths of light, intensities of light, rates, durations, or frequencies for configuring oral device; and
configure the oral device based on the oral device configuration.

14. The system of claim 13, wherein the oral device configuration is generated by a machine learning model based on the sensed data.

15. The system of claim 14, wherein the machine learning model is trained based on cohort data.

16. The system of claim 13, wherein the processor is further configured to:
apply the sensed data as an input to a machine learning model; and
receive a machine learning output from the machine learning model based on the sensed data, the machine learning output comprising the oral device configuration.

17. The system of claim 13, wherein the processor is further configured to:
transmit the sensed data over a network; and
receive the oral device configuration from the network.

18. The system of claim 13, further comprising an analytics module comprising a machine learning model configured to generate the oral device configuration based on the sensed data.

19. The system of claim 13, further comprising an external component, wherein the processor is further configured to:
receive an external component configuration based on the sensed data; and
transmit the external configuration component to the external component.

20. The system of claim 13, wherein the sensed data is one or more of biometric data, exhaled breath condensate (EBC) data, pH levels, saliva data, chemical data, shape data, object data, food data, electrical signal, tooth data, mitochondria data, protein data, glucose data, lactate data, urea data, serum data, blood data, light data, biochemical data, electrochemical data, volatile organic compounds (VOCs) biomarker data, laser data, force data, or movement data.
